(19) United States
(12) Patent Application Publication
(10) Pub. No.: US 2018/0037562 A1
(43) Pub. Date: Feb. 8, 2018

(54) Process for Preparing 5-(Chloromethyl)furfural

(71) Applicant: Far Eastern New Century Corporation, Taipei City (TW)

(72) Inventors: Ruey-Fen LIAO, Taipei City (TW); Xin-An LU, Taipei City (TW); Tzu-Chiao HUANG, Taipei City (TW); Fang-Yu LIU, Taipei City (TW); Chien-Hung CHOU, Taipei City (TW)

(21) Appl. No.: 15/421,500

(22) Filed: Feb. 1, 2017

(30) Foreign Application Priority Data
Aug. 2, 2016 (TW) 105124410

(51) Int. Cl. C07D 307/50; C12P 17/04

(52) U.S. Cl. CPC C07D 307/50; C12P 17/04; C12P 2201/00

(57) ABSTRACT

Disclosed herein is a process for preparing 5-(chloromethyl)furfural which includes the steps of: subjecting a cellulosic biomass to a pretreatment so as to obtain a pretreated cellulosic biomass, the pretreatment including a dilute acid treatment and a steam explosion treatment conducted after the dilute acid treatment; mixing the pretreated cellulosic biomass with hydrochloric acid so as to obtain a mixture; reacting the pretreated cellulosic biomass with the hydrochloric acid in the mixture so as to produce a reaction product containing 5-(chloromethyl) furfural; and extracting 5-(chloromethyl)furfural from the reaction product with an organic solvent.

### CROSS-REFERENCE TO RELATED APPLICATION

This application claims priority of Taiwanese Patent Application No. 105124410, filed on Aug. 2, 2016.

### FIELD

The disclosure relates to a process for preparing 5-(chloromethyl)furfural, and more particularly to a process for preparing 5-(chloromethyl)furfural which involves pretreating a cellulosic biomass with dilute acid and steam explosion.

### BACKGROUND

5-(chloromethyl)furfural is a furan derivative having the formula (I):

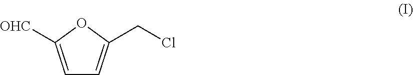

It is known that CMF can be converted to high energy fuels when reacting with a nucleophile, and thus is considered an important fuel precursor. For instance, CMF can be converted into 5-(ethoxymethyl)furfural or 5-methylfurfural when reacting with the respective one of ethanol and hydrogen.

CMF can be prepared by subjecting a hexose (such as glucose and fructose) and hydrochloric acid to a conversion reaction including dehydration and chlorination. However, such conversion process requires a large amount of hexose. In order to replace hexose with an alternative cost-effective starting material and to resolve the environmental problem caused by biomass wastes, converting biomass wastes into high energy fuels such as CMF is becoming the most important research direction. A cellulosic biomass, a common biomass waste, is a renewable energy resource and is abundantly produced from industry and agroforestry operations. Therefore, researchers in this field endeavor to develop a method for preparing CMF by virtue of a cellulosic biomass.

U.S. Pat. No. 7,829,732 B2 discloses a method for preparing CMF or a derivative thereof. Specifically, the method includes contacting cellulose, concentrated hydrochloric acid and 1,2-dichloroethane (DCE) in a reaction vessel at a temperature of about 65° C., so as to form a biphasic mixture with the cellulose and concentrated hydrochloric acid forming an aqueous layer and the DCE forming an organic layer. Thereafter, by heating the biphasic mixture, the cellulose is converted into CMF which is then extracted from the aqueous layer into the organic layer. The organic layer is continuously removed to an isolation vessel. Additional DCE is continuously added into the reaction vessel, and additional CMF is extracted from the aqueous layer into the organic layer thus formed. Thus, a desired amount of CMF or a derivative thereof can be prepared. According to the specification of the aforesaid patent, the cellulose employed may be a cellulosic biomass, which may include a wood residue, a paper waste, an agricultural residue and energy crops. In the examples of the aforesaid patent, cellulose and six types of cellulosic biomasses (i.e., filter paper, cotton, newsprint, wood, corn stover and straw) were each used as a substrate to perform a conversion reaction, and the concentrated hydrochloric acid, DCE and lithium chloride (LiCl, serving as a catalyst) were repeatedly added into the reaction vessel. Therefore, the aforementioned method is not only time- and labor-consuming, but also requires a large amount of DCE. Moreover, various undesired by-products (such as furfural derived from a pentose, as well as 5-(hydroxymethyl)furfural (HMF), 2-(2-hydroxyacetyl)furan (HAF) and levulinic acid (LA) derived from a hexose) are also produced during the aforementioned method.

Many researchers have been trying to improve the CMF preparation process in various aspects. For example, as described in Mascal M. et al. (2009), ChemSusChem., 2:859-861, each of glucose, sucrose, cellulose and corn stover was used as a substrate to react with hydrochloric acid and DCE in a closed system. The results reveal that when the conversion reaction is conducted in a closed system, the reaction time can be greatly reduced. In addition, no HMF, HAF and LA were observed in the organic layer, and only little LA was observed in the aqueous layer. The reduced production of HMF and HAF can signify an improvement of the CMF yield.

Chen Z. Z. et al. investigated the effect of various catalysts upon the one-pot conversion of sugarcane bagasse into CMF. To be specific, sugarcane bagasse was subjected to a reaction with hydrochloric acid and DCE, in which a respective one of ten metal chlorides (i.e., AlCl₃, LiCl, NaCl, KCl, MgCl₂, CaCl₂, BaCl₂, CrCl₃, NiCl₂ and FeCl₃) was added as a catalyst to determine the catalytic efficiency. The results reveal that AlCl₃ leads to a significantly higher CMF yield compared to other metal chlorides. However, when a larger amount of AlCl₃ is used, more CMF degrades, thereby causing a reduction in the CMF yield (Chen Z. Z. et al. (2012), Transactions of the Chinese Society of Agricultural Engineering, 28:214-219).

WO 2014/066746 A1 discloses a method for producing 5-(halomethyl)furfural, particularly 5-(chloromethyl)furfural, by acid-catalyzed conversion of biomass. The aforesaid method includes use of an organic solvent with temperature-dependent solubility for 5-(halomethyl)furfural, which allows for temperature-dependent phase separation of the 5-(halomethyl)furfural from the reaction mixture. In the examples, CMF solubility in various solvents at various temperatures was investigated. The results reveal that CMF is relatively soluble in DCE, toluene and chlorobenzene even at low temperatures. Hexylbenzene, pentylbenzene and dodecylbenzene (including hard type and soft type) were observed to have relatively low solubility for CMF at low temperatures, but relatively high solubility for CMF at elevated temperatures. Particularly, CMF was observed not very soluble in soft type dodecylbenzene at room temperature, while as the temperature increased, CMF was observed to go from a solid/liquid biphasic mixture in soft type dodecylbenzene to a single phase.

Furthermore, according to the disclosure of WO 2014/066746 A1, the biomass employed may be subjected to a pretreatment to help make the sugars in the biomass more accessible (by disrupting the crystalline structures of cellulose and hemicellulose and breaking down the lignin structure). Suitable pretreatments include mechanical treatments, treatments with concentrated acid, dilute acid, SO₂, alkali, and hydrogen peroxide, wet-oxidation, steam explosion, ammonia fiber explosion (AFEX), supercritical CO₂ explosion, treatments with liquid hot water, and organic solvent treatments.

In spite of the aforesaid existing techniques, there is still a need in the art to develop an efficient process of converting a biomass into CMF.

### SUMMARY

Therefore, the disclosure provides a process for preparing 5-(chloromethyl) furfural, which includes the steps of: subjecting a cellulosic biomass to a pretreatment so as to obtain a pretreated cellulosic biomass, the pretreatment including a dilute acid treatment and a steam explosion treatment conducted after the dilute acid treatment; mixing the pretreated cellulosic biomass with hydrochloric acid so as to obtain a mixture; reacting the pretreated cellulosic biomass with the hydrochloric acid in the mixture so as to produce a reaction product containing 5-(chloromethyl) furfural; and extracting 5-(chloromethyl) furfural from the reaction product with an organic solvent.

Other features and advantages of the disclosure will become apparent with reference to the following detailed description and the exemplary embodiments.

### DETAILED DESCRIPTION

Unless defined otherwise, all technical and scientific terms used herein have the meaning commonly understood by a person skilled in the art to which the present disclosure belongs.

One skilled in the art will recognize many methods and materials similar or equivalent to those described herein, which could be used in the practice of the present disclosure. Indeed, the present disclosure is in no way limited to the methods and materials described.

In order to increase the yield of CMF converted from a cellulosic biomass, the applicants found by research that before conducting the conversion reaction, by subjecting the cellulosic biomass first to a dilute acid treatment and subsequently to a steam explosion treatment, or further to an enzymatic hydrolysis treatment after the steam explosion treatment, the CMF yield can be efficiently increased without using any catalyst in the conversion reaction. Therefore, the present disclosure provides a process for preparing CMF, which includes the steps of:

subjecting a cellulosic biomass to a pretreatment so as to obtain a pretreated cellulosic biomass, the pretreatment including a dilute acid treatment and a steam explosion treatment conducted after the dilute acid treatment;

mixing the pretreated cellulosic biomass with hydrochloric acid so as to obtain a mixture;

reacting the pretreated cellulosic biomass with the hydrochloric acid in the mixture so as to produce a reaction product containing 5-(chloromethyl) furfural; and

extracting 5-(chloromethyl)furfural from the reaction product with an organic solvent.

As used herein, the terms “cellulosic biomass” and “lignocellulosic biomass” can be used interchangeably, and refer to any materials containing cellulose.

According to the present disclosure, the cellulosic biomass may be derived from a single source, or may include a mixture derived from more than one source. Suitable cellulosic biomasses include, but are not limited to, bioenergycrops, agricultural residues, municipal solid wastes, industrial solid wastes, sludge from paper manufacture, yard wastes, wood wastes, forestry wastes, and combinations thereof.

In certain embodiments, the cellulosic biomass may be selected from the group consisting of miscanthus, softwood, hardwood, corn cobs, crop residues (such as corn husks), corn stover, grasses, wheat straw, barley straw, hay, rice straw, switchgrass, waste paper, sugarcane bagasse, sorghum plant material, soybean plant material, components obtained from milling of grains, stems, roots, leaves, wood chips, sawdust, shrubs, vegetables, fruits, flowers, and combinations thereof. In an exemplary embodiment of this disclosure, the cellulosic biomass is rice straw. In another exemplary embodiment of this disclosure, the cellulosic biomass is corn stover.

According to this disclosure, via the dilute acid treatment, the hemicellulose contained in the cellulosic biomass may be hydrolyzed to xylose and xylose oligomers. The operating procedures and conditions for the dilute acid treatment are within the expertise and routine skills of those skilled in the art. Examples of dilute acid include, but are not limited to, sulfuric acid, phosphoric acid, hydrochloric acid and nitric acid. In certain embodiments, the dilute acid treatment may be conducted using a 0.2% to 5% (wt) sulfuric acid solution. In certain embodiments, the dilute acid treatment may be performed at a temperature ranging from 110° C. to 160° C. for 30 minutes to 120 minutes. In an exemplary embodiment of this disclosure, the dilute acid treatment is conducted by mixing with a 1% (wt) sulfuric acid solution and heating at 150° C. for 75 minutes. In another exemplary embodiment of this disclosure, the dilute acid treatment is conducted by mixing with a 1% (wt) sulfuric acid solution and heating at 120° C. for 180 minutes.

According to this disclosure, via the steam explosion treatment, the structures of the lignin and cellulose contained in the cellulosic biomass may be disrupted, so as to increase the exposed surface area of the cellulose. The operating procedures and conditions for the steam explosion treatment are within the expertise and routine skills of those skilled in the art. In certain embodiments, the steam explosion treatment may be conducted by heating at a temperature ranging from 170° C. to 210° C. in a stem-containing reaction system for 1 minute to 10 minutes, followed by quickly dropping the pressure of the system to a pressure ranging from 1 atm to 20 atm. In an exemplary embodiment of this disclosure, the steam explosion treatment is conducted by heating at 200° C. in a stem-containing reaction system for 3 minutes to 5 minutes, followed by quickly dropping the pressure of the system to 1 atm.

In certain embodiments, the pretreatment may further includes a solid-liquid separation treatment conducted between the dilute acid treatment and the steam explosion treatment, so as to remove a liquid portion of the dilute acid-treated cellulosic biomass.

According to this disclosure, xylose, xylose oligomers and acetic acid produced during the dilute acid treatment may be removed via the solid-liquid separation treatment, so as to prevent the production of furfural and levulinic acid (LA). The solid-liquid separation treatment is performed using technology well known to a skilled artisan, including but not limited to, filtration, centrifugation and decantation. In an exemplary embodiment of this disclosure, the solid-liquid separation treatment is a filtration treatment.

According to this disclosure, the pretreated cellulosic biomass in the mixture may contain glucan in an amount from 0.1 g to 30 g per 100 mL of hydrochloric acid. In certain embodiments, the pretreated cellulosic biomass in the mixture contains glucan in an amount from 1 g to 8 g per 100 mL of hydrochloric acid. In an exemplary embodiment of this disclosure, the pretreated cellulosic biomass in the mixture contains glucan in an amount of 1 g per 100 mL of hydrochloric acid.

In certain embodiments, the pretreatment may further include an enzymatic hydrolysis treatment using cellulase, which is conducted after the steam explosion treatment.

According to this disclosure, via the enzymatic hydrolysis treatment using cellulose, the cellulose contained in the cellulosic biomass may be hydrolyzed to glucose. The operating procedures and conditions for the enzymatic hydrolysis treatment using cellulose are within the expertise and routine skills of those skilled in the art. In certain embodiments, the enzymatic hydrolysis treatment may be conducted with an enzymatic mixture composed of cellulase and hemicellulase under stirring at a temperature ranging from 50° C. to 60° C. for 48 hours to 96 hours. In an exemplary embodiment of this disclosure, the enzymatic hydrolysis treatment is conducted with the enzymatic mixture composed of cellulase and hemicellulase under stirring at 50° C. for 72 hours.

According to this disclosure, after the enzymatic hydrolysis treatment, the pretreated cellulosic biomass in the mixture may contain glucose in an amount from 0.1 g to 30 g per 100 mL of hydrochloric acid. In certain embodiments, after the enzymatic hydrolysis treatment, the pretreated cellulosic biomass in the mixture contains glucose in an amount from 1 g to 20 g per 100 mL of hydrochloric acid. In an exemplary embodiment of this disclosure, after the enzymatic hydrolysis treatment, the pretreated cellulosic biomass in the mixture contains glucose in an amount of 1 g per 100 mL of hydrochloric acid.

According to this disclosure, the mixing step may be conducted under stirring at a temperature ranging from 10° C. to 35° C. for 40 minutes to 150 minutes. In certain embodiments, the mixing step may be conducted under stirring at a temperature ranging from 10° C. to 35° C. for 20 minutes to 60 minutes. In an exemplary embodiment of this disclosure, the mixing step is conducted under stirring at 25° C. for 40 minutes. In another exemplary embodiment of this disclosure, the mixing step is conducted under stirring at 25° C. for 60 minutes.

The hydrochloric acid according to this disclosure may have a concentration ranging from 4 M to 12 M. In certain embodiments, the hydrochloric acid may have a concentration ranging from 8 M to 12 M. In an exemplary embodiment of this disclosure, the hydrochloric acid has a concentration of 12 M.

According to this disclosure, the organic solvent may be added to the mixture before the reacting step.

The organic solvent according to this disclosure may be selected from the group consisting of dichloromethane, chloroform, 1,2-dichloroethane (DCE), 1,1,2-trichloroethane, chlorobenzene, toluene, amylbenzene, hexylbenzene, dodecylbenzene, and combinations thereof. In an exemplary embodiment of this disclosure, the organic solvent is DCE.

In certain embodiments, the reacting step may be conducted in a closed environment.

In certain embodiments, the reacting step may be conducted by heating at a temperature ranging from 85° C. to 100° C. In an exemplary embodiment of this disclosure, the reacting step is conducted by heating at 100° C.

According to this disclosure, the heating may be conducted for 20 minutes to 60 minutes. In certain embodiments, the heating may be conducted for 40 minutes to 60 minutes. In an exemplary embodiment of this disclosure, the heating is conducted for 40 minutes. In another exemplary embodiment of this disclosure, the heating is conducted for 60 minutes.

The disclosure will be further described by way of the following examples. However, it should be understood that the following examples are solely intended for the purpose of illustration and should not be construed as limiting the disclosure in practice.

### Examples

### General Experimental Procedure:

1. Measurement of the glucan and glucose contents:

The glucan and glucose contents in the test samples were measured using a high performance liquid chromatography (HPLC) system (DIONEX Ultimate 3000) equipped with a refractive index detector (RI detector) according to the laboratory analytical procedures (LAPs) developed by National Renewable Energy Laboratory (NREL). The column and operation conditions for HPLC are as follows: Aminex HPX-87H column (BioRad, length: 250 mm, ID: 4.6 mm); mobile phase: 5 mM sulfuric acid; flow rate: 0.6 mL/min; and sample injection volume: 20 μL.

Furthermore, glucose solutions with different concentrations (0.25-24 mg/mL) were used as control standards.

### 2. Measurement of the CMF Content:

The CMF content in the test samples was measured using a HPLC system (DIONEX Ultimate 3000) equipped with an ultraviolet absorption spectrometer. The column and operation conditions for HPLC are as follows: C18 column (SunFire, length: 250 mm, ID: 4.6 mm); mobile phase: acetonitril/0.05% phosporic acid (10:90, v/v); and sample injection volume: 10 μL. Gradient elution with the mobile phase was conducted for 24.3 minutes as follows: acetonitril was maintained at 10% during 0-3 minutes, was increased from 10% to 30% during 3-4 minutes, was increased from 30% to 50% during 4-11 minutes, was increased from 50% to 100% during 11-11.1 minutes, was maintained at 100% during 11.1-14.1 minutes, was reduced from 100% to 10% during 14.1-14.2 minutes, and was maintained at 10% during 14.2-24.3 minutes. The flow rate of the mobile phase was maintained at 1.0 mL/min during 0-14.2 minutes, was increased from 1.0 mL/min to 1.2 mL/min at 14.2 minute, was maintained at 1.2 mL/min during 14.2-22.3 minutes, was reduced from 1.2 mL/min to 1.0 mL/min at 22.3 minute, and was maintained at 1.0 mL/min during 22.3-24.3 minutes.

Furthermore, CMF solutions with different concentrations (0.25-25 mg/mL) were used as control standards.

### Example 1. Production of CMF from a Cellulosic Biomass Pretreated with Dilute Acid and Steam Explosion

In this example, cellulosic biomasses (including rice straw and corn stover) were each subjected to a dilute acid treatment, followed by a steam explosion treatment. The pulps thus obtained were used to produce CMF under various experimental conditions, so as to investigate the effect of these treatments (for pretreating the cellulosic biomasses) on the CMF yield.

### Experimental Procedures:

The rice straw and corn stover were cut into pieces with a knife, followed by pulverization with a pulverizer. A portion of the pulverized product of rice straw was divided into 8 groups including two control groups (i.e., Control Groups 1-2) and six experimental groups (i.e., Experimental Groups 1-6). A portion of the pulverized product of corn stover served as Experimental Group 7. Each group was subjected to a pretreatment and a conversion reaction as shown in Table 1.

The pretreatment for Experimental Groups 1-6 was described in more detail as follows. Firstly, the pulverized product of rice straw was subjected to a dilute acid treatment with a 1% (wt) sulfuric acid solution, followed by heating at 150° C. for 75 minutes. Then, the resultant mixture was placed in a filter bag having an aperture size of 37 μm (Yichang-filter company, Taiwan, Cat. No. PP 60350S), followed by pressing with a vertical press (RESI Co., Ltd.) at a pressure of 8 Mpa. A solid portion thus collected was placed in a steam explosion reactor system (LUCKY SEVEN INDUSTRIAL Co., Ltd.), into which steam was introduced, and was heated at 200° C. for 3 to 5 minutes. Thereafter, the pressure of the steam explosion reactor system was sharply dropped to 1 atm so as to conduct a steam explosion treatment. The pulp thus obtained served as the pretreated cellulosic biomass.

The pretreatment for Experimental Group 7 is similar to that for Experimental groups 1-6, except that the dilute acid treatment was conducted by heating at 120° C. for 180 minutes.

The pretreatment for Control Group 1 is described in more detail as follows. Specifically, the pulverized product of rice straw was subjected to an alkaline treatment with a 2% (wt) sodium hydroxide solution, followed by heating at 100° C. for 60 minutes. Then, the resultant mixture was subjected to filtration using a filter having an aperture size of 5 μm (ADVANTEC). A solid portion thus obtained served as the pretreated cellulosic biomass.

The pretreatment for Control Group 2 is described in more detail as follows. Specifically, the pulverized product of rice straw was placed in the steam explosion reactor system, into which ammonia was introduced, and was heated at 145° C. for 20 minutes. Thereafter, the pressure of the steam explosion reactor system was sharply dropped to 1 atm so as to conduct an ammonia fiber explosion treatment. The pulp thus obtained served as the pretreated cellulosic biomass.

Afterwards, each group of the pretreated cellulosic biomass was subjected to HPLC according to the method set forth in section 1 of “General Experimental Procedures” so as to determine the glucan content. A suitable amount of the pretreated cellulosic biomass was added into 50 mL of a 12 M hydrochloric acid (HCl) solution such that the resultant mixture had a glucan concentration as shown in Table 1. After pre-agitating the mixture at room temperature for a given time as shown in Table 1, 100 mL of DCE was added to the mixture, followed by incubation in a closed environment. A conversion reaction was conducted at 100° C. for a given time as shown in Table 1. The resulting reaction product was then cooled to room temperature in an ice bath, and an organic layer was separated therefrom by virtue of a separating funnel. The organic layer thus obtained was subjected to HPLC according to the method set forth in section 2 of “General Experimental Procedures” so as to determine the CMF content.

The CMF yield was calculated using the following Equation (I):

A=(B/C)×100 (I)

where
A=the CMF yield (%)
B=the CMF content detected in the organic layer (g/mL)
C=the glucan content in the mixture before the conversion reaction (g/mL)

TABLE 1

| | | | Conditions for conversion reaction | | |
|---|---|---|---|---|---|
| | | | Glucan | | |
| | | | concentration | | |
| | | | in the mixture | | |
| | | | before the | Pre- | |
| | | | conversion | agitation | Reaction |
| | Cellulosic | | reaction | time | time |
| Group | biomass | Pretreatment | (g/100 mL of HCl) | (min) | (min) |
| Experimental | Rice | Dilute acid | 1 | 40 | 40 |
| Group 1 | straw | treatment | | | |
| Experimental | Rice | and steam | 1 | 80 | 40 |
| Group 2 | straw | explosion | | | |
| Experimental | Rice | treatment | 1 | 120 | 40 |
| Group 3 | straw | | | | |
| Experimental | Rice | | 1 | 120 | 60 |
| Group 4 | straw | | | | |
| Experi4ental | Rice | | 1 | 150 | 40 |
| Group 5 | straw | | | | |
| Experimental | Rice | | 1 | 120 | 40 |
| Group 6 | straw | | | | |
| Experimental | Corn | | 2 | 120 | 40 |
| Group 7 | stover | | | | |
| Control | Rice | Alkaline | 1 | 120 | 40 |
| Group 1 | straw | treatment | | | |
| Control | Rice | Ammonia fiber | 1 | 120 | 40 |
| Group 2 | straw | explosion | | | |
| | | treatment | | | |

### Results:

The CMF yield of each of Experimental Groups 1 to 7 and Control Groups 1 to 2 is shown in Table 2.

TABLE 2

| Group | CMF yield (%) |
|---|---|
| Experimental Group 1 | 68 |
| Experimental Group 2 | 66 |
| Experimental Group 3 | 84 |
| Experimental Group 4 | 58 |
| Experimental Group 5 | 75 |
| Experimental Group 6 | 75 |
| Experimental Group 7 | 60 |
| Control Group 1 | 54 |
| Control Group 2 | 43 |

It can be seen from Table 2 that the CMF yield of each experimental group is significantly higher as compared with Control Groups 1 and 2. The experimental results reveal that the CMF yield can be effectively enhanced by subjecting the cellulosic biomass first to a dilute acid treatment and subsequently to a steam explosion treatment before the conversion reaction no matter what the cellulosic biomass is.

### Example 2. Production of CMF from a Cellulosic Biomass Pretreated with Dilute Acid, Steam Explosion and Enzymatic Hydrolysis

In this example, the cellulosic biomasses (including rice straw and corn stover) were each subjected to a dilute acid treatment, a steam explosion treatment and an enzymatic hydrolysis treatment in sequence. The pulps thus obtained were used to produce CMF under various experimental conditions, so as to investigate the effect of these treatments (for pretreating the cellulosic biomasses) on the CMF yield.

### Experimental Procedures:

Another portion of the pulverized product of rice straw as prepared in Example 1 was divided into 10 groups including Control Group 3 and nine experimental groups (i.e., Experimental Groups 8-16). Another portion of the pulverized product of corn stover as prepared in Example 1 served as Experimental Group 17. Each group was subjected to a pretreatment and a conversion reaction as shown in Table 3.

The pretreatment for Experimental Groups 8-16 is similar to that for Experimental groups 1-6, except that an enzymatic hydrolysis treatment was further conducted after the steam explosion treatment. Specifically, the pulp obtained from the steam explosion treatment was subjected to HPLC according to the method set forth in section 1 of “General Experimental Procedures” so as to determine the glucan content. After adjusting the pH value of the pulp to 5 with a 98% NaOH solution, the pulp was subjected to the enzymatic hydrolysis treatment using an enzymatic mixture (Novozymes Cellic® CTec2 which is composed of cellulase and hemicellulase; dosage: 15 FPU per gram of glucan) under agitation at 150 rpm and 50° C. for 72 hours. The resulting hydrolysis product was concentrated under vacuum, and the residue thus obtained served as the pretreated cellulosic biomass.

The pretreatment for Experimental Group 17 is similar to that for Experimental Groups 8-16, except that the dilute acid treatment was conducted by heating at 120° C. for 180 minutes.

The pretreatment for Control Group 3 is similar to that for Control Group 2, except that an enzymatic hydrolysis treatment as employed for Experimental Groups 8-16 was further conducted after the steam explosion treatment. The residue thus obtained served as the pretreated cellulosic biomass.

Afterwards, each group of the pretreated cellulosic biomass was subjected to HPLC according to the method set forth in section 1 of “General Experimental Procedures” in order to determine the glucose content. A suitable amount of the pretreated cellulosic biomass was added into 50 mL of 12 M HCl such that the resultant mixture had a glucose concentration as shown in Table 3. After pre-agitating the mixture at room temperature for a given time as shown in Table 3, 100 mL of DCE was added to the mixture, followed by incubation in a closed environment. A conversion reaction was conducted at 100° C. for a given time as shown in Table 3. The resulting reaction product was then cooled to room temperature in the ice bath, and an organic layer was separated therefrom by virtue of the separating funnel. The organic layer thus obtained was subjected to HPLC according to the method set forth in section 2 of “General Experimental Procedures” so as to determine the CMF content.

The CMF yield was calculated using the following Equation (II):

D=(E/F)×100 (II)

where
D=the CMF yield (%)
E=the CMF content detected in the organic layer (g/mL)
F=the glucose content in the mixture before the conversion reaction (g/mL)

TABLE 3

| | | | Conditions for conversion reaction | | |
|---|---|---|---|---|---|
| | | | Glucose | | |
| | | | concentration | | |
| | | | in the mixture | | |
| | | | before the | Pre- | |
| | | | conversion | agitation | Reaction |
| | Cellulosic | | reaction | time | time |
| Group | biomass | Pretreatment | (g/100 mL of HCl) | (min) | (min) |
| Experimental | Rice | Dilute acid | 1 | 40 | 60 |
| Group 8 | straw | treatment, | | | |
| Experimental | Rice | steam | 1 | 80 | 60 |
| Group 9 | straw | explosion | | | |
| Experimental | Rice | treatment, | 1 | 120 | 20 |
| Group 10 | straw | and | | | |
| Experimental | Rice | enzymatic | 1 | 120 | 40 |
| Group 11 | straw | hydrolysis | | | |
| Experi4ental | Rice | treatment | 1 | 120 | 60 |
| Group 12 | straw | | | | |
| Experimental | Rice | | 1 | 150 | 60 |
| Group 13 | straw | | | | |
| Experimental | Rice | | 5 | 80 | 60 |
| Group 14 | straw | | | | |
| Experimental | Rice | | 10 | 80 | 60 |
| Group 15 | straw | | | | |
| Experimental | Rice | | 20 | 80 | 60 |
| Group 16 | straw | | | | |
| Experimental | Corn | | 1 | 80 | 40 |
| Group 17 | stover | | | | |
| Control | Rice | Ammonia fiber | 1 | 120 | 40 |
| Group 3 | straw | explosion | | | |
| | | treatment and | | | |
| | | enzymatic | | | |
| | | hydrolysis | | | |
| | | treatment | | | |

### Results:

The CMF yield of each of Experimental Groups 8 to 17 and Control Group 3 is shown in Table 4.

TABLE 4

| Group | CMF yield (%) |
|---|---|
| Experimental Group 8 | 71 |
| Experimental Group 9 | 85 |
| Experimental Group 10 | 45 |
| Experimental Group 11 | 75 |
| Experimental Group 12 | 76 |
| Experimental Group 13 | 73 |
| Experimental Group 14 | 65 |
| Experimental Group 15 | 52 |
| Experimental Group 16 | 50 |
| Experimental Group 17 | 85 |
| Control Group 3 | 40 |

It can be seen from Table 4 that the CMF yield of each experimental group is higher as compared with Control Group 3. The experimental results reveal that the CMF yield can be effectively enhanced by subjecting the cellulosic biomass to a dilute acid treatment, a steam explosion treatment and an enzymatic hydrolysis treatment in sequence before the conversion reaction no matter what the cellulosic biomass is. Particularly, the CMF yield of Experimental Group 9 is significantly higher than that of each of Experimental Groups 14-16, thereby indicating that the mixture before the conversion reaction, when containing 1 g of glucose per 100 mL of HCl, gives rise to a better CMF yield.

In view of the foregoing, when the cellulosic biomass is pretreated first with dilute acid and subsequently with steam explosion, or further with enzymatic hydrolysis, the pretreated cellulosic biomass can be efficiently converted into CMF under a suitable conversion reaction.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

1. A process for preparing 5-(chloromethyl)furfural, comprising the steps of:
subjecting a cellulosic biomass to a pretreatment so as to obtain a pretreated cellulosic biomass, the pretreatment including a dilute acid treatment and a steam explosion treatment conducted after the dilute acid treatment;
mixing the pretreated cellulosic biomass with hydrochloric acid so as to obtain a mixture;
reacting the pretreated cellulosic biomass with the hydrochloric acid in the mixture so as to produce a reaction product containing 5-(chloromethyl)furfural; and
extracting 5-(chloromethyl)furfural from the reaction product with an organic solvent.

2. The method of claim 1, wherein the pretreatment further includes an enzymatic hydrolysis treatment using cellulase which is conducted after the steam explosion treatment.

3. The method of claim 1, wherein the pretreatment further includes a solid-liquid separation treatment conducted between the dilute acid treatment and the steam explosion treatment, so as to remove a liquid portion of the cellulosic biomass

4. The method of claim 1, wherein the mixing step is conducted under stirring at a temperature ranging from 10° C. to 35° C. for 40 minutes to 150 minutes.

5. The method of claim 1, wherein the pretreated cellulosic biomass in the mixture contains glucanin an amount from 0.1 g to 30 g per 100 mL of hydrochloric acid.

6. The method of claim 2, wherein the pretreated cellulosic biomass in the mixture contains glucose in an amount from 0.1 g to 30 g per 100 mL of hydrochloric acid.

7. The method of claim 1, wherein the hydrochloric acid has a concentration ranging from 4 M to 12 M.

8. The method of claim 1, wherein the organic solvent is added to the mixture before the reacting step.

9. The method of claim 1, wherein the organic solvent is selected from the group consisting of dichloromethane, chloroform, 1,2-dichloroethane, 1,1,2-trichloroethane, chlorobenzene, toluene, amylbenzene, hexylbenzene, dodecylbenzene, and combinations thereof.

10. The method of claim 1, wherein the reacting step is conducted in a closed environment.

11. The method of claim 1, wherein the reacting step is conducted by heating at a temperature ranging from 85° C. to 100° C.

12. The method of claim 11, wherein the heating is conducted for 20 minutes to 60 minutes.

13. The method of claim 11, wherein the cellulosic biomass is selected from the group consisting of miscanthus, softwood, hardwood, corn cobs, crop residues, corn stover, grasses, wheat straw, barley straw, hay, rice straw, switchgrass, wastepaper, sugarcane bagasse, sorghum plant material, soybean plant material, components obtained from milling of grains, stems, roots, leaves, wood chips, sawdust, shrubs, vegetables, fruits, flowers, and combinations thereof.
